(19) United States
(12) Patent Application Publication
(10) Pub. No.: US 2023/0149003 A1
(43) Pub. Date: May 18, 2023

(54) Cardiac and Vascular Access and Closure System and Method

(71) Applicants: Regents of the University of Colorado, Denver, CO (US); ConneX Biomedical, Inc., Aurora, CO (US)

(72) Inventors: Max Bannister Mitchell, Castle Pines, CO (US); Jeremy H. Morgan, Missoula, MT (US)

(73) Assignees: Regents of the University of Colorado, Denver, CO (US); ConneX Biomedical, Inc., Aurora, CO (US)

(21) Appl. No.: 18/050,955

(22) Filed: Oct. 28, 2022

Related U.S. Application Data
(60) Provisional application No. 63/272,938, filed on Oct. 28, 2021.
(60) Provisional application No. 63/298,980, filed on Jan. 12, 2022.

(51) Int. Cl. A61B 17/00

(52) U.S. Cl. CPC A61B 17/0057; A61B 2017/00579; A61B 2017/00601; A61B 2017/00654

(57) ABSTRACT

Devices, systems and methods for cardiac and vascular access configured to allow for intracardiac access to conduct medical procedures. The devices, systems and methods are particularly useful in trans-cardiac extra-corporeal membrane oxygenation (ECMO) procedures, ventricular assist procedures, cardiopulmonary bypass, or other medical procedures where intracardiac access may be required.

### CROSS-REFERENCE TO RELATED APPLICATIONS

The present application is related to and claims priority to U.S. Provisional Patent Application Serial Nos. 63/272,938, filed Oct. 28, 2021, and 63/298,980, filed Jan. 12, 2022; and is a continuation-in-part of co-pending U.S. patent application Ser. No. 17/466,619, filed Sep. 3, 2021, which is a continuation of U.S. patent application Ser. No. 17/173,914, filed Feb. 11, 2021, now U.S. Pat. No. 11,123,542 issued Sep. 21, 2021.

### BACKGROUND OF THE INVENTION

The present invention pertains generally to devices, systems and methods for cardiac and vascular access. More particularly, the present disclosure pertains to devices, systems and methods for transcardiac access. Still more particularly, the present disclosure pertains to use of the system, devices and methods of the present disclosure as ports for transcardiac access to the internal cardiac anatomy. Further, the present disclosure pertains to catheters and cannulas for transcardiac placement, access, perfusion, or exchange.

The present disclosure also pertains to devices, systems and methods for extra-corporeal membrane oxygenation (ECMO) veno-venous (VV) access (VV-ECMO). Still more particularly, the present disclosure pertains to use of the system, devices and methods of the present disclosure as a right ventricular assist device (RVAD) in patients with right heart failure who have adequate lung function. Further the devices, system and methods of the present disclosure are capable of use as a left ventricular assist device (LVAD) as well.

ECMO is a form of heart-lung machine that may be is useful to treat severe acute respiratory failure (ARF) when conventional ventilator management is inadequate or even injurious to lung health and potential survival. ECMO machines consist, generally, of tubing, a blood pump and an oxygenator. Patients with respiratory failure due to inflammatory diseases and infectious diseases have potentially reversible disease provided support can be maintained for an adequate time. Patients with chronic and or irreversible lung failure may be supported with ECMO as a bridge to lung transplant. VV-ECMO preserves cardiovascular function by withdrawing blood from the systemic venous circulation, i.e., outflow from the body to the ECMO machine where the blood is oxygenated and carbon dioxide is removed. The blood is then re-infused, i.e., inflow to the body from the ECMO machine, to the venous circulation proximal to the lung parenchyma, and typically into the right atrium.

ARF/ECMO requires large bore venous access via one or more percutaneous cannulas. Conventionally, one or more cannulas are placed via the right internal jugular vein, a femoral vein or both. In some cases concomitant pulmonary hypertension impairs right ventricular function necessitating right ventricular support. In this circumstance, return flow right ventricular dysfunction can be managed by returning the blood directly to the pulmonary artery. Current cannula technology limits the duration that ECMO support can be accomplished due to complications at the cannula entry sites and obligatory patient mobility limitations. In addition, many VV-ECMO cannulation strategies can be limited by recirculation where oxygenated blood from the inflow source is withdrawn from the outflow source before it can transit the pulmonary circulation. This results in inefficient or inadequate oxygen delivery to the systemic circulation.

Velour-wrapped paracorporeal cannulas which are intended for medium to long-term use as right and left ventricular assist devices, i.e., RVAD and/LVAD, respectively, transit the abdominal wall to a cardiac chambers or great vessel. In the clinical setting, para-corporeal Berlin Heart EXCOR (Berlin Heart, The Woodlands, Tex.) cannulas were successfully deployed to the right atrium and pulmonary artery for long-term, i.e., greater than 5 months, for ECMO support in patients with infectious ARF. This strategy facilitated aggressive mobilization and rehabilitation. Such a strategy, however, requires full median sternotomy and cardiopulmonary bypass for both insertion and removal of the Berlin Heart EXCOR cannulas. The Berlin Heart EXCOR cannulas are only approved for pediatric sales and use in the United States.

### SUMMARY OF THE INVENTION

It is an objective of the present disclosure to provide a system for trans-cardiac or intravascular access and closure.

It is a further objective of the present disclosure to provide an access cuff device for trans-cardiac access and closure.

It is another objective of the present invention to provide an access cuff comprising a compression assembly for attaching the access cuff to the heart muscle. The compression assembly includes an annular member having a plurality of tissue anchor openings passing there through and at least one attachment skirt configured to be positioned between the annular member and the heart muscle and which allows tissue anchors to pass through the attachment skirt, axially compress the annular member and the attachment skirt to the heart muscle in a hemostatic manner.

It is a further objective of the present disclosure that the annular member be a unitary or segmented compression ring.

It is still another objective of the present disclosure that the access cuff further includes an access cap configured to removably engage with the annular member, wherein the access cap has a central opening and comprises a seal or valve that allows a catheter, cannula, ventricular assist device, surgical instruments, and/or implantable devices to pass into and through the central opening of the access cap and through the access cuff in a hemostatic manner.

It is still a further objective of the present disclosure to provide a method for establishing transabdominal or transthoracic cardiac or vascular access to conduct procedures within the heart, arterial valves, or vasculature.

It is still yet another objective of the present disclosure to provide a transabdominal or transthoracic multi-lumen cannula for access through the cardiac wall or vascular wall and is configured to pass into and through the access cuff.

It is still a further objective of the present disclosure to provide a tubular graft coupled to the access cuff that is configured to function as a working channel through a central annular opening of the access cuff and permit a catheter, cannula, or other instrumentation to pass into and through tubular graft and central annular opening of the access cuff and into the heart.

It is yet a further objective of the present disclosure to provide a securing collar that secures the tubular graft, cannula, and access cuff together in a hemostatic manner and is removable to allow withdrawal of the cannula from the access cuff.

It is yet another objective of the present disclosure to provide a closure device configured to be used with the access cuff after the cannula is withdrawn from the access cuff.

It is still a further objective of the present disclosure to provide a paracorporeal cannula useful with the access cuff to access a ventricular or atrial chamber or transit a cardiac valve.

It is still another objective of the present invention to provide a paracorporeal catheter having plural lumens configured for blood perfusion both from the heart and returned to the heart after transiting an ECMO machine.

It is yet another objective of the present disclosure to provide a closure device configured to be used with the access cuff.

It is still a further objective of the present disclosure to provide a system including the access cuff device, the paracorporeal catheter, and the closure device.

It is yet a further objective of the present disclosure to provide a method of delivering the access cuff and paracorporeal cannula, conducting VV-ECMO, removing the paracorporeal cannula, and closing the access through the ventricular access cuff with the closure device.

It is still another objective of the present disclosure to provide an assist method for right ventricular assist or left ventricular assist using the access cuff device and the hemostatic plug or cap as disclosed.

### BRIEF DESCRIPTION OF THE FIGURES

FIG. 1 is a perspective view of an access cuff in accordance with the present disclosure.

FIG. 2 is a perspective partially exploded view of the access cuff with engaged tissue anchors and an access cap in accordance with the present disclosure.

FIG. 3 is a perspective view of the access cuff engaged with the access cap in accordance with the present disclosure.

FIG. 4 is a perspective cross-sectional view of the access cuff and its engagement with the access cap and showing an attachment skirt associated with the access cuff in accordance with the present disclosure.

FIG. 5 is a is a diagrammatic view illustrating attachment of the access cuff to cardiac muscle tissue illustrating a access cuff delivery device holding the access cuff.

FIG. 6 is a diagrammatic view illustrating the access cuff affixed to the cardiac muscle tissue.

FIG. 7 is a diagrammatic view illustrating a multi-lumen perfusion cannula passing through the access cap in accordance with the present disclosure.

FIG. 8 is a diagrammatic view illustrating passing the dual lumen perfusion cannula into and through the access cuff and engagement of the access cap with the access cuff as well as blood inflow and outflow through the dual lumen perfusion cannula and into the heart right atrium and right ventricle, respectfully.

FIG. 9 is a diagrammatic view illustrating engagement of a hemostatic plug of cap after withdrawal of the dual lumen perfusion catheter and catheter cap associated therewith.

FIG. 10 is a perspective view of an alternative variant of an assembled access cuff in accordance with the present disclosure.

FIG. 11 is a side-elevational, cross-sectional view of the alternative variant of the assembled access cuff in accordance with the present disclosure.

FIG. 12 is a perspective, partial cut-away view of the alternative variant of the assembled access cuff in accordance with the present disclosure.

FIG. 13 is a perspective exploded view of the alternative variant of the access cuff in accordance with the present disclosure.

FIG. 14 is a perspective view of the alternative variant of the access cuff of the present disclosure, an access cannula, and a retaining collar in accordance with the present disclosure.

FIG. 15 is a perspective view of the alternative variant of the access cuff having the flanged graft portion ligated and sealed in accordance with the method of the present disclosure.

FIG. 16 is a perspective view of the alternative variant of the access cuff of the present disclosure, an access cannula, and an alternative retaining collar in accordance with the present disclosure.

FIG. 17 is a perspective view of the alternative variant of the access cuff having the flanged graft portion ligated and sealed in accordance with the method of the present disclosure.

FIG. 18 is a side elevational view of a cannula configured for transabdominal or transthoracic access through the access cuff of the present disclosure.

FIG. 19 is a diagrammatic cross-sectional view taken along line A-A of FIG. 18.

FIG. 20 is a partially exploded view illustrating transabdominal or transthoracic access through the alternative variant of the access cuff and attachment of a retaining collar in accordance with the present disclosure.

FIG. 21 is a diagrammatic view illustrating attachment of the alternative variant of the access cuff, retaining collar and cannula joined to epicardium of the heart muscle.

### DETAILED DESCRIPTION OF THE PREFERRED EMBODIMENTS

The device, system and methods of the present invention will be described with reference to certain exemplary embodiments thereof. These exemplary embodiments or variants are intended to be illustrative and non-limiting examples of the present invention. The example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments or variants may be embodied in many different forms and that neither should be construed to limit the scope of the disclosure. Those of ordinary skill in the art will understand and appreciate that variations in materials, structure, material properties, and tolerances may be made without departing from the scope of the invention, which is defined only by the claims appended hereto and their range of equivalents. In some example embodiments, well-known processes, well-known device structures, and well-known technologies are not described in detail.

Benefits, other advantages, and solutions to problems have been described herein with regard to specific embodiments or variants. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure.

The scope of the disclosure is accordingly to be limited by nothing other than the appended claims, in which reference to an element in the singular is not intended to mean “one and only one” unless explicitly so stated, but rather “one or more.” It is to be understood that unless specifically stated otherwise, references to “a,” “an,” and/or “the” may include one or more than one and that reference to an item in the singular may also include the item in the plural. All ranges and ratio limits disclosed herein may be combined.

Moreover, where a phrase similar to “at least one of A, B, and C” is used in the claims where A, B, and C refer to claimed elements, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching when used throughout the figures to denote different parts but not necessarily to denote the same or different materials.

For ease of understanding, the present invention is described with reference to the accompanying Figures. In the accompanying Figures like elements are identified by like reference numerals.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s)<r feature(s) as illustrated in the figures. Spatially relative terms may be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than and including totally.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision with some approach to exactness in the value; approximately or reasonably close to the value; nearly. If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

“Transabdominal” is intended to mean passing across the abdominal wall and/or abdominal cavity.

“Transcardiac” is intended to mean passing across the cardiac wall.

“Transthoracic” is intended to mean passing across the chest and/or the thoracic cavity.

Medical or anatomical terms are intended to have their usual and customary meaning in the medial arts and terminology.

The steps recited in any of the method or process descriptions may be executed in any order and are not necessarily limited to the order presented. Furthermore, any reference to singular includes plural embodiments, and any reference to more than one component or step may include a singular embodiment or step. Elements and steps in the figures are illustrated for simplicity and clarity and have not necessarily been rendered according to any particular sequence. For example, steps that may be performed concurrently or in different order are illustrated in the figures to help to improve understanding of embodiments of the present disclosure.

Any reference to attached, fixed, connected or the like may include permanent, removable, temporary, partial, full and/or any other possible attachment option. Additionally, any reference to without contact (or similar phrases) may also include reduced contact or minimal contact. Surface shading lines may be used throughout the figures to denote different parts or areas but not necessarily to denote the same or different materials. In some cases, reference coordinates may be specific to each figure.

Systems, methods, and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.”

As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

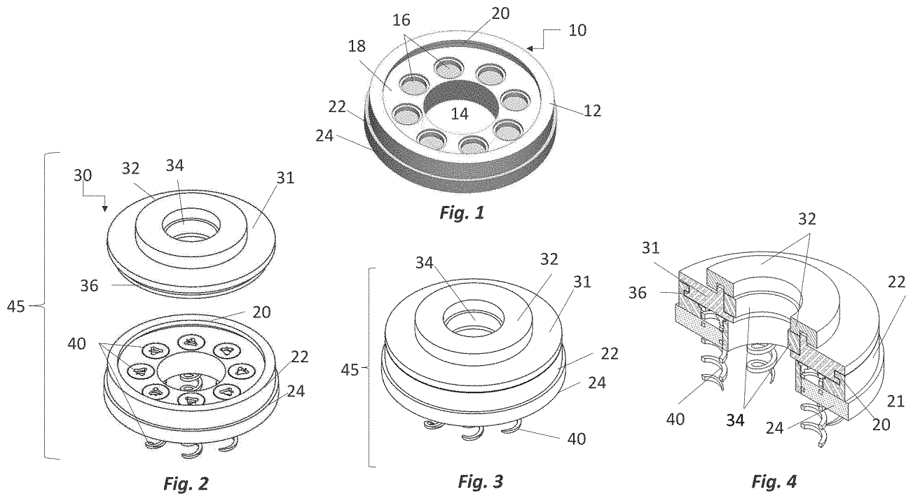

The system 45 of the present disclosure consist generally of several component devices, namely, an access cuff 10 shown in FIG. 1 and FIG. 2, a medical device 150, depicted for reference purposes only as paracorporeal dual lumen perfusion cannula, shown in FIG. 7, an access cap 30 shown in FIGS. 2, 3 and 4, and a hemostatic plug or cap 54 shown in FIG. 9. The access cuff serves as a trans-cardiac access site for the dual lumen perfusion cannula. The access cuff 10 is configured for placement on an epicardial surface of a heart 5, such as the left or right ventricle, and allow access through the cardiac muscle tissue and into the ventricular space to allow passage of the medical device 150, e.g., a dual lumen perfusion cannula, through the access cuff, into the cardiac ventricle and, in the case of the perfusion cannula, extend past the associated arterial valve into the distal associated artery.

The access cuff 10 as shown in FIGS. 1, 2, 3 and 4 is configured for placement onto the epicardial surface through a minimal access incision, such as a subxyphoid epigastric incision or limited thoracotomy. The access cuff 10 is comprised of an annular member 12, having a central bore opening 14, a plurality of tissue anchor apertures 16 passing axially through a flange 18 concentrically surrounding the central bore opening 14, and an attachment skirt 24 having a central opening axially aligned with the central bore opening 14 of the annular member 12.

The annular member 12 has an outer circumferential edge 22 having a circumferential projection 20 projecting axially therefrom and defining an outward aspect of flange 18. The circumferential projection 20 is configured to have a mating attachment 21 that couples to a corresponding mating surface 36 of the access cap 30. The mating attachment 21 and mating surface 36 may take a wide variety of configurations, including, without limitation, friction fit, interference fit, threaded fit, interlocking fit, bayonet fit, press-fit, or the like.

The attachment skirt 24 has a first surface that is positioned to abut flange 18 on a heart-facing surface thereof. A second surface of attachment skirt is positioned to abut the cardia tissue 5. The attachment skirt 24 facilitates hemostasis with surgical attachment to a cardiac surface 5 and may be comprised of a flexible or rigid felt material, such as polytetrafluoroethylene (PTFE), polyethylene terephthalate (PET), or similar non-woven or woven material suitable for creating a hemostatic seal. The attachment skirt 24 has a plurality of tissue anchor receiving sites arrayed about a circumferential aspect of the attachment skirt and is configured to allow for tissue anchors to penetrate into and through the attachment skirt 24 without substantial bleeding.

Tissue anchors 40 engage perpendicularly into and through the tissue anchor receiving sites on the attachment skirt and are deployed either individually, in groups, or simultaneously to couple the access cuff to the ventricular surface. The tissue anchors and attachment skirt exert an axially compressive force to secure the access cuff to the epicardial surface of the ventricle with hemostasis. Optionally, the access cuff has a central cylinder defining the central bore opening.

While the tissue anchors shown in the accompanying Figures are helical coil tissue screws, it will be understood that alternative tissue anchors, including, for example, sutures, barbs, pins, or the like, are also useful as tissue anchors in the present disclosure.

The access cuff 10, tissue anchors 40, and attachment skirt 24 are conceptually similar to the apical cuff attachment system disclosed in commonly owned U.S. Pat. No. 11,123,542 and co-pending, commonly owned U.S. patent application Ser. No. 17/466,619, filed Sep. 2, 2021, which are hereby incorporated by reference in their entirety.

The access cap 30, as shown in FIGS. 2, 3 and 4, is fixedly or detachably coupled to the access cuff 10. The access cap 30 has a first annular member 31configured to mate with the access cuff 10 by engaging the circumferential projection 20 of access cuff 10. To facilitate a hemostatic engagement there between, first annular member may have a mating surface 36 which cooperates with mating attachment 21 of the access cuff 10. A second annular member 32 engages with the first annular member 31 and serves to retain a seal 34 centrally positioned within the central annular opening formed by axial alignment of first annular member 31 and the second annular member 21. Access cap 30 has a central opening 34 configured to allow the medical device 150 to pass through the access cap 30 by passing through the second annular member 32, the seal 34, which seals against the medical device 150, then passing through the first annular member 31 and into and through the access cuff 10 and into the heart 5. Seal 34 is centrally positioned in the access cap 30 that is configured to bear against an outer wall surface of the medical device 15 and both allow the medical device 150 to be axially adjusted through the seal 34 and provide a hemostatic seal around the outer wall surface of the medical device 150 once it is in a desired position. Seal 34 may be a circumferential seal such as a Tuohy-Borst valve or an O-ring seal, or may be a one-way valve such as dome valve, a duck-bill, or multi-cuspid valve seal.

Following patient recovery or transplant, the access cuff is configured accept and secure a hemostatic plug that can be inserted via repeat minimally invasive exposure and coupled to the access cuff to provide a hemostatic seal of the central opening of the access cuff. The access cuff is capable of being produced in a wide variety of sizes and central opening diameters to accommodate a range of cannula and patient sizes.

The medical device 150, shown for example as a paracorporeal dual lumen perfusion cannula useful with the present system is thin-walled and reinforced for kink resistance. Other examples of medical devices 150 which may be used with the access cuff system 45 of the present disclosure include, for example, catheters, cannulas, ventricular assist devices, medical or surgical instruments, endoscopes, implantable devices, conduits, or the like.

For purposes of non-limiting example only and without intent limit the use of system 45 to ECMO procedures, reference is being made with illustration to a multi-lumen perfusion cannulas used in ECMO procedures as medical device 150. An example of an ECMO cannula suitable for use as a medical device with the present system is the PROTEK DUO cannula (LivaNova, Arvada, Colo.) described in U.S. Pat. Nos. 10,279,101, 9,782,534, or 9,168,352, each of which are hereby incorporated by reference.

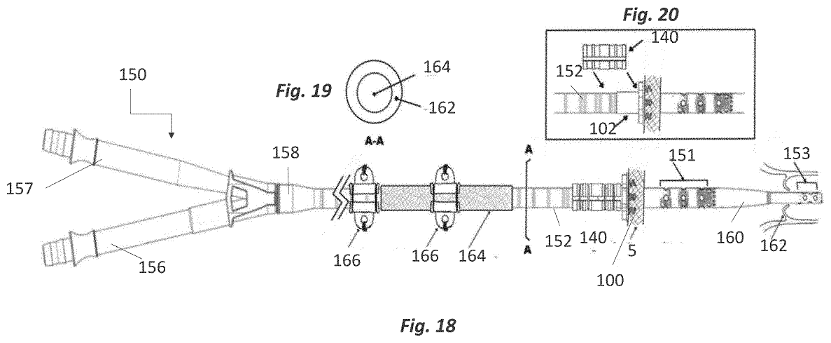

As shown in FIG. 18, in perfusion cannula 150, a first lumen 156 functions as the blood outflow lumen to communicate blood from the patient to the ECMO machine. A second lumen 157 functions as the blood return lumen to return oxygenated blood from the ECMO machine to the patient. Inflow openings 153 communicate with first lumen 156 and are positioned at a distal end 160 of the cannula 160 and transit the cardiac valve 162. The distal end 160 of the cannula 150 is tapered such that it is smaller in diameter than more proximal portions 152 of the cannula 150. Inflow openings 153 may alternatively be positioned at the distal end 160 of the cannula 150 and open axially and/or inflow openings 153 may pass through side wall surfaces of the distal end 160 of the cannula 150 and open generally perpendicular to the longitudinal axis of the cannula 150. The inflow openings 153 are radiographically identifiable and positioned on the distal end of the cannula to sit distal to the cardiac valve 162.

Proximal to the distal end 160 of the cannula 160, both the inflow or second lumen 157 and outflow or first lumen 156 are present within the cannula 150. Cannula 150 is characterized by having a tapered increase in cannula diameter to accommodate the inflow 157 and outflow 156 lumens. The largest cannula diameter is positioned at the position of the outflow lumen 156. At no point do the inflow lumen 157 and outflow lumen 156 communicate blood flow with each other, rather the blood inflow and blood outflow conduits remain separate from each other. The outflow lumen 156 has a section with multiple outflow apertures 151 that permit blood to flow out of the outflow lumen 156, through the outflow apertures 151 and into the ventricular chamber. The outflow apertures 151 are radiographically identifiable.

The portion of the cannula 150 that is secured by the access cuff 10 is configured to permit secure fixation of the cannula 150 without kinking or damage to the outer cannula wall or to the inflow and outflow lumens. The intra-corporeal portion of the cannula 150, i.e., that part of the cannula between the access cuff 10 and the patient's skin where the cannula 150 exits the abdominal wall, may be coated or wrapped with a covering 164 that promotes tissue fixation, such as a velour wrap, and reduce the risk of ascending infection along the cannula course. The portion of the cannula external to the skin is intended to accommodate a fixation mechanism 166 to secure the cannula 150 to the body without cannula kinks. At the proximal most aspect of the cannula 150, the inflow and outflow lumens bifurcate 158 with each lumen forming a larger diameter clampable segment that terminates in an integrated tubing connector intended for connection to ECMO circuit tubing. Each tubing connector accommodates a removable and/replaceable plug to control hemostasis during placement. The inflow lumen plug also accommodates a wire for Seldinger placement of the cannula. The entire length of the cannula 150 is radiopaque.

A hemostatic plug or cap 54, shown in FIG. 9, is provided that operably engages with the access cuff 10 after the medical device 150 and access cap 30 have been removed from the patient. Hemostatic plug or cap 54 provides a hemostatic seal of the central opening 14 of the access cuff 10. The hemostatic plug 54 maintains hemostasis as the patients are weaned from support following recover or transplant to eliminate the need to remove the access cuff 10 from the heart 5. This approach minimizes bleeding risk and greatly facilitates medical device 150 removal via a limited incision. In use, the hemostatic cap or plug 54 is rapidly inserted and securely fixed to the access cuff 10 to occupy and occlude the central opening 14 of the access cuff that was previously occupied by the medical device 150.

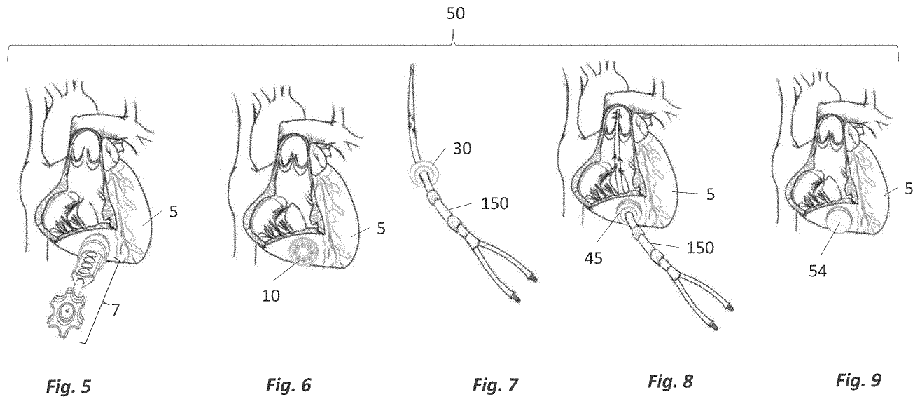

FIGS. 5 to 9 illustrate the method 50 of delivering and using the system 45. In use, the access cuff 10 is delivered, such as by use of a delivery tool 7 that allows for simultaneous driving of the tissue anchors 40. Delivery tool 7 is disclosed in co-pending and commonly assigned U.S. patent application Ser. No. 17/351,082, filed Jun. 17, 2021, which is hereby incorporated by reference in its entirety. As illustrated in FIG. 5, the access cuff 10 is placed against the epicardial surface of the heart 5, and the tissue anchors 40 are driven, either synchronously or individually, into the cardiac tissue 5 to secure the access cuff 10 to the heart tissue and cause the attachment skirt 34 to bear against the cardiac tissue 5 with an axially compressive force to the access cuff 10. The delivery tool 7 illustrated in FIG. 5 may be used to drive the tissue anchors 40 synchronously and then the individual tissue anchors may be adjusted manually. As noted above, the tissue anchors 40 may be helical coil tissue screws, barbs, sutures, or the like.

As illustrated in FIG. 6, once the delivery tool 7 has been used to secure the access cuff 10 to the cardiac tissue 5 and the central bore opening 14 of the access cuff 10 exposes a working channel through the access cuff 10 to the cardiac tissue 5. As illustrated in FIG. 7, the access cap 30 is engaged onto an outer wall surface of the medical device 150 and is preliminary positionally placed along the length of the medical device 150. Alternatively, the access cap 30 may be coupled to the access cuff 10 prior to placement of the medical device 150. A wire is placed into and through the central bore opening 14 of the access cuff 10, such as by using the Seldinger technique. A dilator may be used over a wire to increase the cardiac tissue opening before passing the medical device 150 into and through the cardiac tissue 5 through the central bore opening 14 of the access cuff 10 and access cap 30. Once the distal end medical device 150 is positioned with the heart, and in the case of a perfusion cannula the inflow openings 151 are positioned to open away from the cardiac valve 162, the access cap 30 is sealed against the dual lumen perfusion cannula to achieve hemostasis as shown in FIG. 8.

As shown in FIG. 9, once the procedure is completed, such as by removing the patient from the ECMO machine, the access cap 30 is disengaged from the access cuff 10, and the medical device 150 and access cap 30 are removed and the hemostatic plug or cap 54 is engaged with the access cuff 10 to occlude the central bore opening 14 of the access cuff 10 with hemostasis.

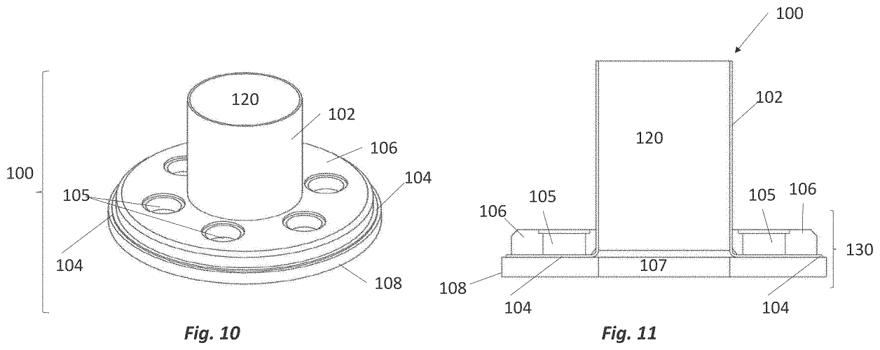

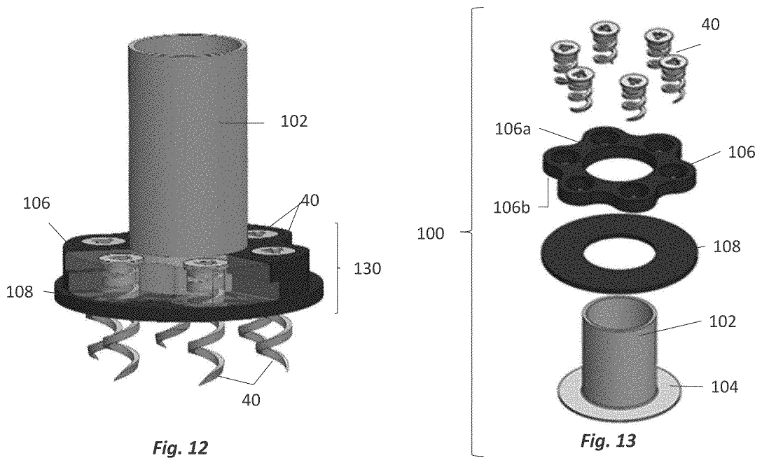

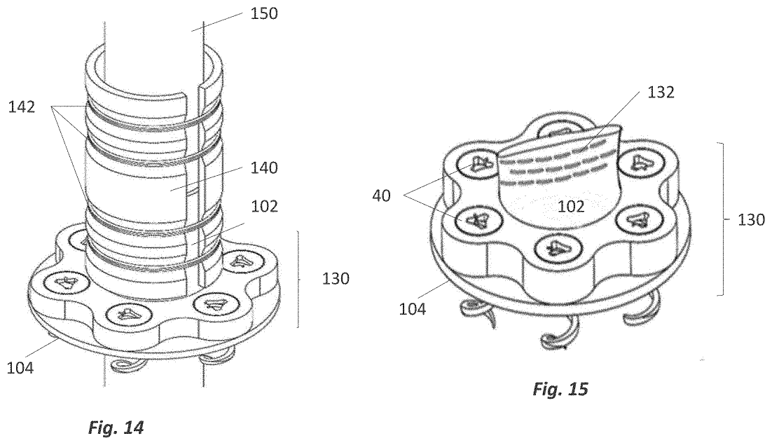

An alternative embodiment or variant of the access cuff system 45 as shown in FIGS. 1-9 is shown in FIGS. 10-21. Turning now to FIGS. 10-21, the alternative access cuff system 100 is shown. Like access cuff system 45, alternative access cuff system 100 includes an axial compression ring 106, which may be unitary or segmented, a plurality of tissue anchor openings 105 arrayed about the entire circumference of the axial compression ring 106, an attachment skirt 108, and a central bore opening 107 passing through the axial compression ring 106, the attachment skirt 108 and a plurality of tissue anchors 40. Like with access cuff system 45, the access axial compression ring 106, attachment skirt 108, and tissue anchors 40 are similar to the axial compression plate, sewing skirt, and tissue anchors disclosed U.S. Pat. No. 11,123,542, issued Sep. 21, 2021 (hereinafter the '542 Patent) which is incorporated herein by reference in their entirety.

In addition, alternative access cuff system 100 includes a flanged tubular graft 102 having a central lumen 120 wherein the central lumen 120 is positioned in axial alignment with the central bore opening 107 and a radial flange 104 of the flanged tubular graft 102 is positioned between the axial compression ring 106 and the attachment skirt 108. Flanged tubular graft 102 is comprised of a pliable graft material and cardiac or vascular access may be established through the central lumen 120. In this configuration, as shown in FIGS. 13 and 13, the tissue anchors 40 pass into and through each of the tissue anchor openings 105, into and through the radial flange 104 and the attachment skirt 108 and into the cardiac tissue 5 (not shown). In this configuration the tissue anchors 40 bear against an upper aspect 106a of the axial compression ring 106 causing an axial force to be applied to the attachment ring 108 and the radial flange 104 of the flanged tubular graft 102. The radial flange 104 of the tubular graft 102 may be positioned between the axial compression ring 103 and the attachment skirt 108, or as alternatively illustrated in FIG. 13, a lower surface 106b of the axial compression ring 106 may be adjacent the attachment skirt 108 and the radial flange 104 may be abutting a distal surface of the attachment skirt 108, with the lumen 120 of the tubular graft 102 extending through the entire central bore opening 107 of the axial compression ring 106 and the attachment skirt 108. Flanged tubular graft 102 may be similar to that disclosed in U.S. patent application Ser. No. 16/739,807, filed Jan. 10, 2020 (hereinafter the '807 Patent Application), which is incorporated herein by reference in their entirety.

Optional second attachment skirt (not shown) may be interposed between a proximal surface of the radial flange 104 of flanged tubular graft 102 and a distal surface 106b the axial compression ring 106 and is concentric with a proximal tubular portion of tubular graft 102. Alternatively or in addition, optional second attachment skirt may be interposed between a distal surface of the radial flange 104 and cardiac muscle tissue 5. The compression assembly 130 may, optionally, be hemostatically integrated with the graft 102 such as, for example, by coating with silicone, reflowing, co-extruded during manufacture of the access cuff system 100, adhesive joining of one or more of the compression ring 106, attachment skirt 108, and radial flange 104.

The at least one attachment skirt 108 facilitates hemostasis with surgical attachment to the heart muscle 5 or vascular tissue and may be made of a flexible or rigid felt material, such as polytetrafluoroethylene (PTFE), polyethylene terephthalate (PET), or similar non-woven or woven material suitable for creating a hemostatic seal.

The tubular graft 102 and radial flange 104 may be a single unitary member or may be a hybrid assembly in which the tubular graft 102 and radial flange 104 are discrete members configured to be removably joined together. In the hybrid assembly, the tubular graft 102 is in whole in part of a rigid or semi-rigid tubular member having a connector, such as threads, snap-fit, interference fits, or the like, positioned at a distal end of the tubular graft. The connector at the distal end of the tubular graft removably engages with a mating connector associated with the radial flange 104.

The central bore opening 107 of the axial compression ring 106 is configured to accommodate the graft 102 to pass through the central opening 107 and has a distal surface 106b configured to bear against the proximal surface of radial flange 14. The plurality of tissue anchor openings 105 pass through the axial compression ring 106 and accommodate a plurality of tissue anchors 40 to pass through the openings 105 and into and through the both the attachment skirt 108 and the radial flange 104 and into the cardiac muscle tissue 5 to axially compress the annular compression assembly 130 against the cardiac muscle tissue 5 in a hemostatic manner. As is described in the '542 Patent, incorporated by reference herein, the axial compression ring 106 may be a unitary ring member or a segmented ring member comprising two or more arcuate segments configured to form a continuous or discontinuous ring structure. Where the axial compression ring 106 is a segmented ring member, the two or more arcuate segments may, optionally, have projections and/or receivers that are configured to interdigitate with adjacent arcuate sections. In this configuration, the interdigitated arcuate members form a flexible annular ring structure that is capable of conforming to different surface topographies on the heart or vascular tissue.

The axial compression ring 106, whether a continuous or discontinuous ring structure, may be made of a rigid biocompatible metal, such as titanium or stainless steel, or a flexible or semi-rigid material such as polyurethane, polyether ether ketone (PEEK) or similar polymeric material. The axial compression ring 106 may also be adhesively joined to the attachment skirt 108.

Each of the plurality of tissue anchor openings 105 in the axial compression ring 106 may optionally have countersunk recesses and/or grommet members lining each of the plurality of openings. The grommet members may line the plurality of openings and, optionally, may have alignment projections that intercalate between the adjacent layers of the compression assembly 130 and assist in providing both alignment and substantially uniform compression and allow the compression assembly 130 to conform to a shape of the cardiac muscle tissue 5 or vascular tissue when joined thereto.

A delivery tool 7 is also provided as part of a system for coupling the axial compression ring 106 to cardiac 5 or vascular tissue. The delivery tool is similar to that described in U.S. Pat. No. 11,338,126 (hereinafter the '126 Patent), which is hereby incorporated by reference in its entirety. In the '126 Patent Application there is described a synchronous drive system to deliver an apical cuff and axial compression ring and synchronously drive tissue anchors into and through an apical cuff and into cardiac muscle tissue to secure an axial compression ring and apical cuff to cardiac muscle tissue. Like the delivery tool and synchronous drive system of the '126 Patent, the delivery system of the present disclosure is capable of operating in a synchronous drive mode to simultaneously drive all of the plurality of tissue anchors 40. However, unlike that delivery and synchronous drive system described in the '126 Patent, the drive system must have a central bore through which the access graft of the present access device will pass and allow the delivery and synchronous drive system to engage the tissue anchors and drive them into the cardiac or venous tissue to axially compress the annular ring and graft thereto. Alternatively, the synchronous drive component may be eliminated and the delivery tool may simply have a delivery housing with a plurality of openings axially aligned with the tissue anchors 40 on the axial compression ring 106 to allow a drive tool to be inserted through each of the plurality of openings to drive each tissue anchor 40 and axially compress the annular ring 106 against the cardiac 5 or venous tissue.

Transabdominal access to heart 5 may be accomplished by a subxiphoid incision with or without partial inferior sternal split with use of a self-retaining retractor, e.g., a Bookwalter retractor. Once access to the heart 5 is established, the delivery tool 7 preloaded with the graft 102 and compression assembly 130, is delivered through the incision, the radial flange 104 is positioned against the heart 5, and the tissue anchors 40 axially driven to pass into and through the axial compression ring 106, the attachment skirt 108, and the radial flange 104 and into the heart muscle 5 thereby axially compressing the compression assembly 130 in a hemostatic manner to the heart muscle 5. As noted above, the tissue anchors 40 may be axially driven synchronously or individually, into the heart muscle 5, preferably without substantial axial force being applied to the tissue anchors 40 themselves but only a rotational force being employed to drive the tissue anchors 40 into the heart muscle 5.

Once the access cuff system 100 is secured to the heart muscle 5 or vascular tissue, transcardiac or transvascular access accomplished through the central lumen 120 of the graft 102 and through either the cardiac muscle 5 or vascular tissue.

Where the two-piece assembly of the tubular graft 102 and removably coupled radial flange 104 is employed, the compression assembly 130 including the radial flange 104 is delivered with the delivery tool, the tissue anchors 40 are driven into the heart muscle 5 of vascular tissue heart muscle 5 in the manner as described above. After the delivery tool is removed, the tubular graft 102 is coupled to the radial flange 104 by engaging the connector on the distal end of the tubular graft 102 with the mating connector on the radial flange 14. Access is then available through the central lumen 120 of the tubular graft 102 and the compression assembly 130.

Once access is no longer required, closure and hemostasis may be achieved by clamping, suturing, and trimming the tubular graft 102 in the case of the unitary tubular graft. In the case of the two-piece assembly, the central opening 107 of the annular ring member 06 and radial flange 104 may be occluded with a cap (not shown) having a connector that mates with the radial flange connector.

In accordance with the present disclosure, there is also provided a medical device 150, such as a perfusion cannula as described above, suitable for use in transabdominal or transthoracic cardiac or vascular access, ECMO, perfusion, exchange, and/or bypass.

At noted previously, at least one attachment skirt 108 is provided at either one surface of radial flange 104 or at both the proximal and distal surfaces of the radial flange 104.

Tissue anchors 40 engage perpendicularly into and through the tissue anchor openings 105 in the axial compression ring 106, the at least one attachment skirt 108, and pass through the radial flange 104, and into the cardiac muscle tissue 5 or vascular tissue. As discussed above, the tissue anchors 40 are deployed either individually, in groups, or simultaneously to couple the compression assembly 130 and the graft 102 to the cardiac muscle 5 or vascular tissue. The tissue anchors 40 exert an axially compressive force to secure the access cuff 10 to the cardiac muscle 5 or vascular tissue with hemostasis. As described in the '126 Patent, the synchronous drive system described therein drives the tissue anchors 40 by application of a rotary force and without substantial axial force applied to the tissue anchors 40. In this manner, rotation of the tissue anchors 40 drives the tissue anchors 40 into the attachment skirt 108 and the cardiac tissue 5.

While the tissue anchors shown 40 in the accompanying Figures are shown as helical coil tissue screws, it will be understood that alternative tissue anchors, including, for example, sutures, barbs, pins, or the like, are also useful as tissue anchors in the present disclosure.

A retaining collar 140 is provided to hemostatically seal the tubular graft 102 circumferentially around the medical device 150 and bridges both the tubular graft 102 and the medical device 150 to circumferentially compress tubular graft 102 against an outer surface of the medical device 150 to create for hemostasis at the juncture there between. Retaining collar 140 has at least one arcuate segment that circumferentially surrounds the graft 102 and the catheter portion 102 of medical device 150. A plurality of circumferential compression rings 142, which may be ligatures, clamps, elastic bands, or the like, compress the at least one arcuate segment 140 against the graft 102 to hemostatically seal inner luminal walls of central lumen 120 of graft 102 against the medical device 150. For example, where the medical device 150 is a perfusion cannula, as shown in FIGS. 18 and 20, the retaining collar 140 is placed over both the tubular graft 102 and the cannula 150 and retained by compression rings 142. The plurality of compression rings 42 are removable to permit removing of the retaining collar 140 and disengagement of medical device 150 from the access cuff 10 when a procedure is completed.

FIG. 18 illustrates engagement of the access cuff system 100, medical device 150, in this case a perfusion cannula, and collar 140 with heart muscle tissue 5.

Once a procedure is completed and the medical device 150 is disengaged from the access cuff system 100, the central lumen 120 of graft 102 is hemostatically sealed such as by surgical staples or sutures 132 and the graft 102 is then trimmed to reduce the length of graft 102, as shown in FIG. 15.

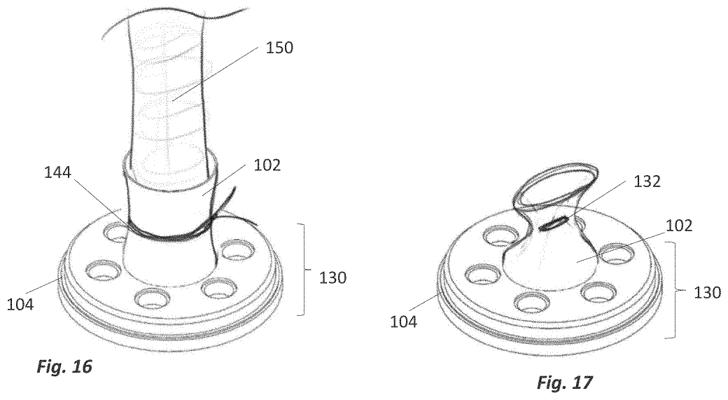

An alternative retaining collar 144 may be employed by employing a suture winding about the tubular graft 102 and the medical device 150 to create hemostasis at the juncture there between as depicted in FIG. 16. Then, after the procedure is completed and the medical device 150 removed from the tubular graft 102, staples or sutures 132 may be used to close the central lumen 120 of the tubular graft 102 and the tubular graft 102 trimmed to a desired length.

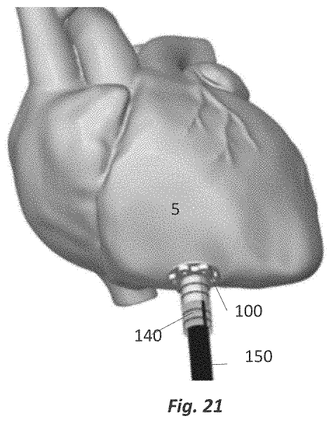

Finally, FIG. 21 depicts the compression assembly 130 with tubular graft 102 (not visible) joined to a medical device 150 by retaining collar 140; all joined to the heart muscle 5.

While the present disclosure is made with reference to certain embodiments or variants, those skilled in the art will understand and appreciate that variations in materials, dimensions, tolerances, material properties, arrangements, procedural steps, and the like are all intended to be within the scope of the disclosure, which is intended to be limited only by the claims appended hereto.

What is claimed is:

1. A system for transabdominal or transthoracic direct cardiac or direct vascular access, comprising:
a compression assembly comprising:
an axial compression ring having a plurality of anchor openings passing axially through the axial compression ring, the axial compression ring have a central axial opening passing therethrough;
an attachment skirt comprising a material capable of imparting hemostasis when compressed against anatomical tissue, the attachment skirt having a central annular opening configured to be in axial alignment with the central axial opening of the axial compression ring when the attachment skirt and the axial compression ring are in an adjacent relationship;
a plurality of tissue anchors configured to cooperate with the plurality of anchor openings, pass through the attachment skirt, and apply an axial compressive force to the axial compression ring, bear against the attachment skirt, and pass into anatomical tissue, thereby axially compressing the compression assembly against the anatomical tissue in a substantially hemostatic manner; and
an access cap configured to sealingly engage with the axial compression ring, the access cap comprising an annular member having a central opening configured to be in axial alignment with the central axial opening of the axial compression ring and the central annular opening of the attachment skirt engaged with the axial compression ring and a seal projecting into the central opening of the annular member.

2. The system of claim 1, wherein the axial compression ring is a unitary ring member.

3. The system of claim 1, wherein the axial compression ring is a segmented ring member.

4. The system of claim 1, wherein the axial compression ring further comprises a plurality of arcuate members.

5. The system of claim 4, wherein the plurality of arcuate members are interdigitated.

6. The system of claim 4, wherein each of the plurality of arcuate members further include at least one of a projection or a recess that interdigitates with at least one of a projection or a recess on an adjacent one of the plurality of arcuate members.

7. The system of claim 1, wherein the axial compression ring is further comprised of a rigid, semi-rigid, or flexible material selected from the group of metals and polymers.

8. The system of claim 1, wherein the tissue anchors further comprise helical tissue screws having a driver opening passing through a tissue screw head and in axial alignment with a central rotational axis of the helical tissue screws.

9. The system of claim 1, wherein the axial compression ring further comprises a projection that mates with a mating projection on the access cap.

10. The system of claim 1, wherein the access cap further comprises a first annular member and a second annular member, wherein the seal is retained between the first annular member and the second annular member.

11. The system of claim 1, further comprising an access graft coupled to the compression assembly.

12. The system of claim 11, wherein the access graft further comprises a tubular graft having a radially projecting flange at one end of the tubular graft.

13. The system of claim 12, wherein the tubular graft passes into and through a central opening of the compression assembly formed by the axial alignment of the central axial opening of the axial compression ring and the central annular opening of the attachment skirt.

14. The system of claim 13, wherein the radially projecting flange is positioned between the axial compression ring and the attachment skirt.

15. The system of claim 12, wherein the access graft further comprises a tubular graft having a first connector and the radially projecting flange further comprises a discrete member having a second connector configured to removably couple with the first connector.

16. The system of claim 1, further comprising a dual lumen cannula configured to pass into and through the central lumen of the access graft, the central opening of the axial compression ring and into cardiac or vascular tissue.

17. The system of claim 1, further comprising a plug configured to couple to the compression assembly in a hemostatic manner.

18. System for transabdominal or transthoracic direct cardiac or direct vascular access, comprising:
an access cuff comprising a tubular graft having a central lumen that communicates between proximal and distal ends of the tubular graft;
a compression assembly comprising a radial flange projecting radially outward from the distal end of the tubular graft, an axial compression ring having a central opening configured to receive the access graft there through, the axial compression ring having a plurality of tissue anchor openings passing axially through the axial compression ring, and at least one attachment skirt joined to and abutting at least one of a distal or proximal surface of the radial flange, the at least one attachment skirt being comprised of a hemostatic material;
a plurality of tissue anchors configured to engage cardiac or vascular tissue and axially compress the compression assembly against cardiac or vascular tissue.

19. The system of claim 18, further comprising an attachment skirt positioned on a distal surface of the radial flange.

20. The system of claim 19, wherein the plurality of tissue anchors in combination with the axial compression ring are configured to exert an axially compressive force about an entire circumference of the attachment skirt against heart or vascular tissue.
